(19) United States
(12) Patent Application Publication
(10) Pub. No.: US 2012/0309822 A1
(43) Pub. Date: Dec. 6, 2012

(54) TREATMENT OF INFECTIOUS DISEASES

(76) Inventor: Peter Blower, London (GB)

(21) Appl. No.: 13/513,372

(22) PCT Filed: Dec. 3, 2010
(86) PCT No.: PCT/GB10/52020

§ 371 Date: Aug. 24, 2012

Related U.S. Application Data
(60) Provisional application No. 61/226,234, filed on Jul. 16, 2009.

(51) Int. Cl. A61K 31/353; A61P 31/04; A61P 31/10; A61P 33/02; A61P 1/00; A61P 31/22; A61P 25/00; A61P 25/28; A61P 31/08; C07D 311/54; A61P 31/00

(52) U.S. Cl. 514/456; 549/399

(57) ABSTRACT

The present invention relates to the treatment or prevention of infectious disorders, and to tonabersat or an analogue of formula (1), and compositions comprising tonabersat or an analogue of formula (1) for use in said treatments.

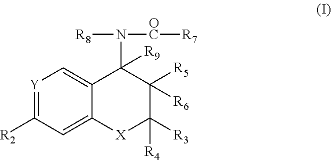

The present invention relates to tonabersat and its analogues and compositions comprising tonabersat or its analogues for use in the treatment of infectious disorders more particularly conditions associated with or resulting from infectious disease or infection with an infective agent.

International patent application WO 95/34545 discloses a series of specific named compounds, including tonabersat, otherwise known as cis-6-acetyl-4-(S)-(3-chloro-4-fluorobenzoylamino)-3,4-dihydro-2,2-dimethyl-2H-1-benzopyran-3-(S)-ol, which is a member of the class of drugs called neuronal gap junction blockers, and which is currently being investigated for a range of conditions including migraine, epilepsy, depression and other neurological conditions.

U.S. Pat. No. 5,948,811 (incorporated herein by way of reference) describes a class of compounds (the analogues of formula I′) which may be used for the prophylaxis and treatment of disorders within the central and peripheral nervous system, including migraine, psychosis, epilepsy and other neurological conditions.

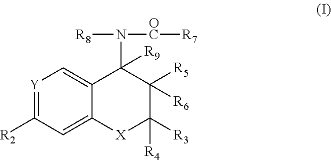

### Y is C—R₁;

R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃ S; or a group CF₃-A-, where A is —CF₂—, —CO—, —CH₂—, CH(OH), SO₂, SO, CH₂—0, or CONH; or a group CF₂ H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇, alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂ X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

Infectious diseases are clinically evident diseases resulting from the presence of pathogenic agents, including pathogenic viruses, pathogenic bacteria, fungi, protozoa, multicellular parasites and aberrant proteins known as prions. A prion is an infectious agent which transmits a mis-folded protein. Prion associated diseases include Creutzfeldt-Jakob disease (CJD), Bovine Spongiform Encephalopathy (BSE) Scrapie Disorder, Gerstmann-Sträussler-Scheinker syndrome (GSS), Fatal familial insomnia (sF1) and Kuru. Collectively these diseases are known as Transmissible Spongiform Encephalophathies (TSEs). An infectious disease is not synonymous with an infection, and may not cause important clinical symptoms or impair host function. However, infectious diseases show a diverse range of symptoms which vary depending on the particular infective agent and result in a wide variety of disorders that may result directly or indirectly from the presence of, or damage caused by an infectious agent.

An infectious disorder, as opposed to an infectious disease, is a disorder resulting directly from the presence of an infectious agent such as prion protein which can lead to CJD, or Human Immunodeficiency Virus which can result in the development of HIV neuralgia, or Streptococcus pneumoniae which can result in Meningitis, or is a disorder resulting indirectly from a current or previous infection or infectious disease, or is a disorder which results from damage caused by an infectious agent such as varicella zoster virus which can result in the development of postherpetic neruralgia. Infectious disorders are often a result of an inflammatory response elicited by the presence of an infectious agent.

There are many treatments available for infectious diseases including anti-viral, anti-bacterial, anti-fungal treatments. These treatments attack the infectious agent and in doing so eventually and hopefully eradicate or control the infectious disease. However there exists a critical need for safe, alternative and improved methods and compositions for the treatment of infectious disorders.

In a first aspect, the present invention provides for tonabersat, or an analogue of formula I,

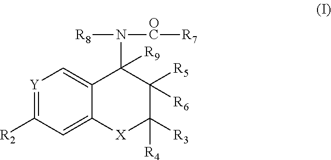

### Y is C—R₁;

R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃ S; or a group CF₃-A-, where A is —CF₂—, —CO—, —CH₂—, CH(OH), SO₂, SO, CH₂ —O, or CONH; or a group CF₂ H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇, alkanoyl; one of R₃ and R₄ is hydrogen or C₁₋₄ alkyl and the other is C₁₋₄ alkyl, CF₃ or CH₂ X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl or a pharmaceutically acceptable composition thereof, for use in the treatment of infectious disorders.

A preferred analogue of formula 1 is the compound carabersat or (trans-(+)-6-acetyl-4-(S)-(4-fluorobenzoylamino)-3,4-dihydro-2,2-dimethyl-2 H-1-benzo[b]pyran-3R ol,hemihydrate.

For therapeutic administration according to the present invention, tonabersat or an analogue of formula I, is most preferably employed in the form of its free base, but may also be used in the form of a pharmaceutically acceptable salt, preferably the hydrochloride salt. Alternative salts with pharmaceutically acceptable acids may also be utilised in prophylactic and/or therapeutic administration, for example salts derived from acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All references to tonabersat or an analogue of formula I, herein includes all pharmaceutically acceptable salts, and all solvates thereof.

Also included within the scope of the present invention are polymorphs, solvates and radiolabelled derivatives of tonabersat or an analogue of formula I, and pharmaceutically acceptable compositions thereof. References to tonabersat or an analogue of formula I, include such polymorphs, solvates and radiolabelled derivatives thereof.

For prophylactic and/or therapeutic administration according to the invention, tonabersat or an analogue of formula I, may be administered in pure form, but will preferably be formulated into any suitable pharmaceutically acceptable and effective composition which provides effective levels of the active ingredient in the body.

Accordingly, the present invention provides a method for the treatment of infectious disorders, comprising administering to a patient in need thereof a pharmaceutically effective amount of tonabersat or an analogue of formula I, or a pharmaceutically acceptable composition thereof.

The present invention also provides for tonabersat, or an analogue of formula I, or a pharmaceutically acceptable composition thereof, for use in the manufacture of a medicament for the treatment of infectious disorders.

By infectious disorders we mean:
i) disorders resulting directly from the presence of pathogenic microbial agents, such as viruses, bacteria, fungi, protozoa or prions. Such disorders include bacterial disorders such as Meningitis from Streptococcus pneumoniae. Such disorders also include prion related disorders such as CJD, BSE, Scrapie Disorder, Gerstmann-Sträussler-Scheinker syndrome (GSS), Fatal familial insomnia (sF1) and Kuru. Collectively these diseases are known as Transmissible Spongiform Encephalophaties (TSEs).
ii) disorders resulting indirectly from a current or previous infection or infectious disease, such as chronic fatigue syndrome, peptic ulcer.
iii) disorders resulting from damage caused by an infectious agent such as one of the herpes viruses including varicella zoster virus, herpes simplex, Epstein-Barr virus and cytomegalovirus which can result in the development of postherpetic neruralgia, HIV which causes extensive damage to the nervous system and results in a severe form of neuropathic pain called HIV neuralgia. Diphtheria and leprosy are bacterial diseases characterized by extensive peripheral nerve damage, and Lyme disease which can cause a wide range of neuropathic disorders.

All treatments may be acute or prophylactic. For acute treatment, a rapid onset of action is preferred, and therefore, drugs that reach maximum plasma concentrations shortly after administration would be most beneficial. Accordingly, compositions providing rapid drug-release and/or dissolution are preferred.

Tonabersat or an analogue of formula I, may be delivered alone, but will generally be delivered in the form of a pharmaceutically acceptable composition thereof, which comprises tonabersat or an analogue of formula I, and one or more pharmaceutically acceptable diluents or carriers selected with regard to the intended route of administration.

Treatment with tonabersat or an analogue of formula I, or a pharmaceutically acceptable composition thereof, may be conducted at a unit dose of between 1 to 1000 mg, suitably 1 to 500 mg, for example an amount in the range of from 2 to 400 mg such as 2, 5, 10, 20, 30, 40, 50, 80, 100, 200, 300 and 400 mg of the active compound.

Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

Preferably, the tonabersat or an analogue of formula I, or a pharmaceutically acceptable salt thereof, is administered to the patient at dose ranges of approximately 0.01 to 15 mg/kg/day, more usually 0.1 to 6 mg/kg/day, for example 1 to 6 mg/kg/day.

It is preferred that tonabersat or an analogue of formula I, is administered in the form of a pharmaceutical composition, such as a composition for oral, including sub-lingual, intranasal, rectal, topical, parenteral (especially intravenous), ocular or aural administration.

Pharmaceutical compositions suitable for the delivery of tonabersat or an analogue of formula I, and methods for their preparation will be readily apparent to those skilled in the art. Such compositions and methods for their preparation may be found, for example, in Remington's Pharmaceutical Sciences, 19th Edition (Mack Publishing Company, 1995).

Compositions suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Liquid formulations may also be prepared by the reconstitution of a solid, for example, from a sachet.

Orally administrable compositions may be in the form of oral solid compositions, such as tablets, capsules, pastilles, pellets, pills, lozenges powders and granules. The composition may be in solid form which melts on contact with the tongue of the patient, for example in the form of disintegrating tablets sold under the trade name ZYDIS®. Shaped oral compositions are preferred, since they are more convenient for general use.

Solid forms for oral administration are usually presented in a unit dose, and contain conventional additives such as adjuvants, binding agents, diluents, disintegrants, dispersing agents, excipients, fillers, tabletting agents, lubricants, colorants, flavourings, desiccants, humectants, and wetting agents.

Pills, pellets and tablets may be coated according to well known methods in the art. Oral solid formulations also include conventional sustained release formulations, such as tablets or granules having an enteric coating.

Suitable fillers include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions are prepared by admixture, and may be prepared by conventional methods of blending, filling, tabletting or the like. Repeated blending operations may be used to distribute the active agent throughout those compositions employing large quantities of fillers. Such operations are, of course, conventional in the art.

They may also be in the form of oral fluid preparations, including liquid preparations, such as aqueous or oily blends, mixtures, suspensions, solutions, emulsions, syrups, tinctures and elixirs, and gel preparations.

They may also be presented as dilutable fluid concentrates or dry product reconstitutable powders for dilution or reconstitution with water or other suitable vehicle before use.

Oral fluid preparations, including gels and liquid preparations may contain conventional additives such as: suspending agents, for example sorbitol, syrup, methyl cellulose, gelatin, hydroxyethylcellulose, carboxymethyl cellulose, aluminium stearate gel or hydrogenated edible fats; emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid; and, if desired, conventional flavouring or colouring agents.

Compositions for oral administration may be formulated to be immediate and/or modified release. Modified release formulations include delayed-, sustained-, pulsed-, controlled-, targeted and programmed release.

Compositions suitable for parenteral administration include injectable and infusible aqueous or oily blends, mixtures, suspensions, solutions, emulsions and low-viscosity gel preparations. Compositions for parenteral administration may be formulated to be immediate and/or modified release. Modified release formulations include delayed-, sustained-, pulsed-, controlled-, targeted and programmed release.

The parenteral compositions for use in the invention may be prepared as long acting depot preparations. Such formulations may be administered by intramuscular injection. Thus, for example, the composition of the invention may be formulated with suitable polymeric or hydrophobic materials (for example as an emulsion in a pharmaceutically acceptable oil) or ion exchange resins, or as sparingly soluble derivatives, for example, as a sparingly soluble salt.

Advantageously, adjuvants such as a local anaesthetic, preservatives and buffering agents are also dissolved, emulsified or suspended in the vehicle.

Such compositions are prepared by admixture of the compound and a solvent or vehicle. The compound, depending on the vehicle and the concentration, can be emulsified, suspended or dissolved. Parenteral compositions are normally prepared by with the compound and a vehicle which is sterile, and/or the composition is sterilised, before filling into a suitable vial or ampoule and sealing.

To enhance the stability, the composition may also be presented as a dry product reconstitutable powder for reconstitution with water or other suitable vehicle before use. A fluid composition can be frozen after filling into the vial and freeze-dried under vacuum.

Tonabersat or an analogue of formula I, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™ Bioject™, etc.) injection.

Formulations for topical administration may be formulated to be immediate and/or modified release. Modified release formulations include delayed-, sustained-, pulsed-, controlled-, targeted and programmed release.

Compositions for use in surgical wounds may be prepared as long acting depot preparations. Such formulations may be administered by implantation (for example subcutaneously or intramuscularly) or by intramuscular injection. Thus, for example, the composition of the invention may be formulated with suitable polymeric or hydrophobic materials (for example as an emulsion in an acceptable oil) or ion exchange resins, or as sparingly soluble derivatives, for example, as a sparingly soluble salt.

It is more preferred that tonabersat or an analogue of formula (I) is administered in the form of a unit-dose composition for administration into a temporary or permanent human or other animal bodily orifice, such as the trachea, nostril, nasal passage, rectum, udder duct, urethra or vagina, or a surgical would, e.g. an incision, or any device inserted in such a temporary or permanent orifice, such as a catheter, trochar, cannula, endotracheal or other endoscopic tube or an ostomy tube, e.g. a tracheostomy or colostomy tube. Intranasal administration is greatly preferred

Intranasally mucosal administrable compositions may be in the form of intranasal mucosal solid compositions, such as powders and granules. They may also be in the form of intranasal mucosal fluid preparations, including liquid preparations, such as aqueous or oily blends, mixtures, suspensions, solutions, emulsions and elixirs, and gel preparations. They may also be presented as dilutable fluid concentrates or dry product reconstitutable powders for dilution or reconstitution with water or other suitable vehicle before use.

Tonabersat or an analogue of formula I, may be administered intranasally or by inhalation, typically in the form of a dry powder from a dry powder inhaler (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

Solid forms for intranasal mucosal administration are usually presented in a unit dose, and contain conventional additives, such as adjuvants, diluents, dispersing agents, excipients, colorants, desiccants, humectants, and wetting agents.

Powders and granules may be coated according to well known methods in the art. Intranasal mucosal solid formulations also include conventional sustained release formulations, such as powders or granules having a resistant coating.

Suitable excipients include cellulose, mannitol, lactose, chitosan, pectin, cellulose derivatives such as hydroxypropylmethylcellulose, methyl cellulose, hydroxyethylcellulose, carboxymethyl cellulose, polyoxamers, such as poly(ethylene oxides), gelatin, polyvinylpyrrolidone and starch. Suitable pharmaceutically acceptable wetting agents include sodium lauryl sulphate.

Solid intranasal mucosal compositions are prepared by admixture, and may be prepared by conventional methods of blending or the like. Repeated blending operations may be used to distribute the active agent throughout those compositions employing large quantities of excipients. Such operations are, of course, conventional in the art.

Intranasal mucosal fluid preparations, including gels and liquid preparations may contain conventional additives such as suspending agents, for example sorbitol, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional colouring agents.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise an analogue of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Tonabersat or an analogue of formula I, may also be administered rectally or vaginally, for example, in the form of a suppository, pessary, or enema.

Tonabersat or an analogue of formula I, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compositions of tonabersat or an analogue of formula I, may also be in the form of fast-dispersing dosage forms such as those described in Expert Opinion in Therapeutic Patents, 11 (6), 981-986, by Liang and Chen (2001) and Verma R K et. al. Current Status of Drug Delivery Technologies and Future Directions, Pharmaceutical Technology On-Line, 2001, 25(2), 1-14. Such dosage forms are also known as oral fast-dissolving, rapid-dissolve, rapid-melt, mouth-dissolving and fast-disintegrating tablet. The composition may be in solid form which melts on contact with the tongue of the patient, for example in the form of disintegrating tablets sold under the trade name ZYDIS® (RP Scherer, UK). Alternatively, the composition may be in the form of the EFVDAS (effervescent drug absorption system, Elan Corporation), Fast Melt (highly porous microfine matrix tablet, Elan Corporation), Flashdose (floss matrix utilising shearform technology, (Fuisz Technologies, USA), Flashtab (orodispersible multiparticulate tablet, Prographarm, France), Multiflash (fast disintegrating multi-unit, multiparticulate tablet, Prographarm), Orasolv (effervescent dispersed microcapsule tablet, Cima Labs Inc, USA), Wowtab tablets (Yamanouchi Pharma Technologies, USA), LYOC (freeze dried fast dispersing tablets, Farmalyoc, France) or Quicksolve (freeze dried fast dispersing tablets, Janssen Pharamceutica, USA).

Other suitable formulation technologies may include INDAS (insoluble drug absorption system, Elan Corporation), which utilises a stabilised amorphous form of the drug with enhanced solubility, NanoCrystal technology (Elan Corporation), which utilises nanoparticles of the drug, typically having a particle size of less than 400 nm in diameter, or SoftGel (RP Scherer), which utilises a soft gelatin capsule formulation.

The formulation technologies described herein may advantageously provide more rapid drug dissolution and absorption. For those compositions that disintegrate in the oral cavity, such as beneath the tongue, the rate of absorption may be increased and first-pass metabolism effects reduced.

As is common practice, the compositions will usually be accompanied by written or printed directions for use in the medical treatment concerned.

The compositions for use in the invention may contain from 0.1% to 99% by weight, preferably from 1%-60% by weight, of the active material, depending on the method of administration.

The following are given by way of example only to illustrate and aid understanding of the invention:

Studies with tonabersat have employed a number of different formulations including:
Direct compression tablets 0.05, 1.0, 10 and 25 mg with tablet core weight 250 mg
Direct compression tablets 15, 25, 40 and 80 mg with tablet core weight 400 mg
Direct compression tablets 20 mg with core weight 400 mg
Nanoparticulate tablets 10, 20 and 40 mg with tablet core weight 400 mg

The direct compression tablets utilise micronized drug substance whilst the nanoparticulate tablets were direct compression tablets utilising wet bed milled spray dried nanoparticulate drug substance. Clinical trials have been conducted utilising 10, 20, 30, 40, 60 and 80 mg round white uncoated direct compression tablets with a core weight of 400 mg with the following unit composition (20 mg tablet only presented; all other strengths differ only in tonabersat and lactose content):

A representative formulation suitable for use in the present invention is detailed in Table 1.

TABLE 1

| | 20 mg Tablet |
|---|---|
| Ingredient | Quantity (mg) |
| Tonabersat | 20.0 |
| Lactose | 330.0 |
| Microcrystalline Cellulose | 20.0 |
| Sodium Starch Glycollate, Type A | 24.0 |
| Colloidal Silicon Dioxide | 2.0 |
| Magnesium Stearate | 4.0 |
| Total Weight | 400.0 |

### EXPERIMENTAL METHODS

### Example 1

### Investigation of Prion Neuroinflammation in Mice

### Aim

To investigate the effect of tonabersat on two models of prion neuropathogenesis: prion infected mice using mock-inoculated mice as controls; and Pneumococcus induced brain inflammation, as a model of inflammation.

### Materials and Method

1. C57b16N mice are used as a model of scrapie and BSE strain prion replication (intracerebral injection of 10⁶ LD50 prion infectivity). They are compared to mock-inoculated similar mice. Acute and chronic injection of Tonabersat is done, at doses ranging from 0.5 mg/kg to 10 and 20 mg/kg (in saline or phosphate buffers solution, with i.p. or p.o. administration). The group is composed of 10 animals for EEG analysis and 10 animals for behavioural test and mortality. Analyses include, during the incubation period (at the first third, the second third and the last third):
(a) EEG relative power analysis (EEG spectral powers were calculated as the ratio of the spectral power obtained per minute in the treatment recording session over the spectral power obtained in the vehicle recording treatment), comparing infected and non-infected mice. ANOVA procedure determines the significance of the results. These tests also include the comparison with drugs that modify the EEG signals or behaviour of the rodents (imipramine, venlafine, diazepam, amphetamine, fluoxetine, caffeine, chlorpromazine, buspirone, at pharmacological doses, i.e. generally from 1 to 32 mg/kg, in NaCL or PBS), with or without gap junction modulators (Tonabersat). That helps evaluate the level of relation between gap junctions and specific pathological neurotransmitter systems, most of which are damaged during the infection.
(b) Behavioural tests: forced swim (i.e. Porsolt test or behavioutal despair), Open Field, Activity Meter, Tail Suspension Test, Dark Light Box. Tests are also made on mice injected with drugs that induce behaviour changes. T test helps determine the significance of the results.
(c) Mortality and morbidity are evaluated and significance is evaluated using Kaplan-Meier estimator.
(d) Histological analyses are made to evaluate the localisation of inflammation (using GFAP and Vimentin labellings) and connexins.
2. Meningitis (and therefore neuroinflammation) is triggered in mice using a pathogenic bacteria, Streptococcus pneumoniae, at 105 CFU (Colony Forming Unit), in the right cerebral hemisphere. Similar parameters as those concerning prion infected mice will be evaluated and compared to antibiotic-treated infected mice (the antibiotic will be chosen among ceftriaxone, vancomycin, cefotaxim, cefdinir at pharmacological doses.
3. Mouse glial and neuronal cell lines infectable by scrapie strain prion (PrPres Biochemical quantification) are used for prion susceptibility investigations. Effects of Tonabersat given before or after infection (22L prion strain 1⁰³, 1⁰⁴, 1⁰⁵ LD50, in triplicate in six well plate format) are investigated. PrPres will be quantified after 3 passages. The toxicity of Tonabersat in these cellular models is investigated prior to study initiation (a dose-ranging study from 1 nM to 100 mM will be done at this time). Other gap junction inhibitor will be tested as well (meclofenamic acid, glycyrrhetinic acid, fluflenamic acid, after dose-rangind studies).

1. Tonabersat or an analogue of formula 1 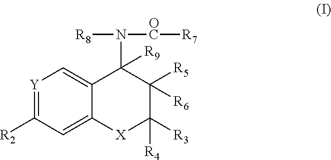
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃ S; or a group CF₃-A-, where A is —CF₂—CO—, —CH₂—, CH(OH), S0₂, SO, CH₂—O, or CONH; or a group CF₂ H-A′- where A′ is oxygen, sulphur, SO, S0₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂ X^{a} is fluoro, chloro, bromo, iodo, alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ON0₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C. sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or N R₁₂ where R₁₂ is hydrogen or C₁₋₆ alkyl or a pharmaceutically acceptable composition thereof, for use in the treatment of infectious disorders where the infectious disorder results from the presence of bacteria, fungi, protozoa, prions or herpes viruses.

2. Tonabersat or an analogue of formula 1 as defined in claim 1, in the manufacture of a medicament for use in the treatment of infectious disorders where the infectious disorder results from the presence of bacteria, fungi, protozoa, prions or herpes viruses.

3. A method for the treatment or prevention of infectious disorders, comprising administering to a patient in need thereof a pharmaceutically effective amount of tonabersat or an analogue of formula 1 as defined in claim 1 where the infectious disorder results from the presence of bacteria, fungi, protozoa, prions or herpes viruses.

4. The method of claim 3 wherein the infectious disorder results from the presence of bacteria.

5. The method of claim 3 wherein the infectious disorder results from the presence of herpes viruses.

6. The method of claim 5 wherein the disorder is postherpetic neuralgia.

7. The method of claim 3 wherein the infectious disorder results from the presence of prions.

8. The method of claim 7 wherein the disorder is a Transmissible Spongiform Encephalopathy.

9. The method of claim 8 wherein the disorder is Creutzfeldt-Jakob disease.

10. The method of claim 8 wherein the disorder is Gerstmann-Straussler-Scheinker syndrome.

11. The method of claim 8 wherein the disorder is Fatal familial insomnia.

12. The method of claim 8 wherein the disorder is Kuru disease.

13. The method of claim 4, wherein the bacteria is Streptococcus pneumoniae.

14. The method of claim 4, wherein the bacteria causes diphtheria, leprosy or Lyme disease.

15. The method of claim 3 wherein the infectious disorder causes chronic fatigue syndrome or peptic ulcers.
